(19) United States
(12) Patent Application Publication
(10) Pub. No.: US 2007/0248667 A1
(43) Pub. Date: Oct. 25, 2007

(54) Tannate Compositions and Methods of Use

(76) Inventor: Jeffrey H. Ping, Cumming, GA (US)

Correspondence Address: SUTHERLAND ASBILL & BRENNAN LLP, 999 PEACHTREE STREET, N.E., ATLANTA, GA, 30309, US

(21) Appl. No.: 11/771,343

(22) Filed: Jun. 29, 2007

Related U.S. Application Data
(63) Continuation of application No. 10/862,119, filed on Jun. 4, 2004, now abandoned.
(60) Provisional application No. 60/476,300, filed on Jun. 5, 2003.

(51) Int. Cl. A61K 31/34; A61K 9/20; C07D 489/02

(52) U.S. Cl. 424/464; 514/282; 514/468

(57) ABSTRACT

Compositions comprising an opiate tannate such as, but not limited to, hydrodocone tannate or codeine tannate, alone or in combination with one or more additional active ingredients from the antihistamine, decongestant, expectorant, and/or antitussive categories which are effective when administered orally for the symptomatic relief of cough associated with respiratory tract conditions such as the common cold, bronchial asthma, acute and chronic bronchitis are disclosed.

### CROSS REFERENCE TO RELATED APPLICATIONS

This application claims priority to U.S. Provisional Application Ser. No. 60/476,300 filed on Jun. 5, 2003.

### FIELD OF INVENTION

The invention relates to novel methods and compositions for extended symptomatic treatment of cough associated with respiratory tract conditions such as the common cold, bronchial asthma, acute and chronic bronchitis.

### BACKGROUND OF INVENTION

Tannate compositions, for the treatment of upper respiratory symptoms associated with respiratory tract conditions such as the common cold, bronchial asthma, acute and chronic bronchitis, are widely used. Such tannate compositions consist of various combinations of active ingredients in the tannate form from the antihistamine, decongestant, expectorant, and or antitussive classes.

The opium group of narcotic drugs are among the most powerfully acting and clinically useful drugs producing depression of the central nervous system. Drugs of this group are used principally as analgesics, but possess numerous other useful properties such as cough suppression. Narcotic analgesics and antitussives, including hydrocodone and codeine, exert their primary effect on the central nervous system and gastrointestinal tract.

Hydrocodone is a semisynthetic opioid antitussive and analgesic with multiple actions qualitatively similar to those of codeine. The precise mechanism of action of hydrocodone and other opiates is not known; however, hydrocodone is believed to act directly on the cough center. Hydrocodone is known chemically as 4, 5α-epoxy-3-methoxy-17-methylmorpinan-6-one.

Codeine is an opiate antitussive that suppresses the cough reflex by a direct effect on the cough center in the medulla and appears to exert a drying effect on respiratory tract mucosa and to increase viscosity of bronchial secretions. Codeine is known chemically as 7, 8-didehydro-4, 5α-epoxy-3-methoxy-17-methylmorpinan-6α-ol.

Tannate salts are typically prepared by reacting the drug free base with tannic acid in the presence of a volatile solvent, such as isopropanol, or water and then vacuum or freeze drying. Reaction variables such as mixing time and temperatures vary depending on the drug molecule and solvent used. Other methods of tannate preparation include the mixing of solid free base with solid tannic acid under heated conditions until completely converted to the tannate salt.

A considerable number of tannic acids occur in nature. Chemically, these acids are described as polymers of different hydroxybenzoic acids. Generally, when the term tannic acid is employed, as in the present case, the acid referred to is gallotannic acid. The internal ester of gallic acid also frequently referred to as tannin.

Tannic acid consists of an amorphous powder, glistening scales, or spongy masses varying in color from yellowish-white to light brown. Tannic acid is very soluble in water or alcohol.

Commercially available tannic acid (Spectrum Chemical in Gardena, Calif.), also known as tannin, has a complex non-uniform chemistry, usually contains from about 5% to about 10% water by weight, has a molecular weight of about 1700, and is typically produced from Turkish or Chinese nutgall.

### DETAILED DESCRIPTION OF THE INVENTION

The present invention provides a therapeutic composition for the symptomatic relief of cough associated with respiratory tract conditions such as the common cold, bronchial asthma, and acute and chronic bronchitis in warm-blooded animals in need of such treatment, said composition comprising a pharmaceutically effective amount of an opiate tannate and a pharmaceutically acceptable carrier.

In preferred embodiments, the opiate tannate is hydrocodone tannate. In preferred embodiments, the pharmaceutically effective amount of the opiate tannate is about 1 to 60 mg of hydrocodone tannate, or about 15 mg of hydrocodone tannate. In other preferred embodiments, the opiate tannate is codeine tannate. In preferred embodiments, the pharmaceutically effective amount of the opiate tannate is about 1 to 120 mg of codeine tannate, or about 30 mg of codeine tannate. The invention provides compositions for the manufacture of a medicament for the treatment of the above conditions comprising the same.

The invention provides that the novel use of opiate tannate compounds, such as but not limited to hydrocodone tannate and codeine tannate, and novel combinations comprising these opiate tannates, produces a therapeutic composition possessing extended antitussive properties. Hydrocodone and codeine suppress the cough reflex by depressing the medullary cough center. The precise mechanism of action of hydrocodone, codeine, and other antitussive opiates is not known, although it is believed to relate to the existence of opiate receptors in the central nervous system.

The present invention is directed to methods and compositions for treating upper respiratory indications in humans and animals, both adult and juvenile, comprising administration of compositions comprising opiate tannates, such as hydrocodone tannate or codeine tannate, alone or in combination with one or more therapeutic agents. Such therapeutic agents include, but are not limited to, therapeutically effective amounts of tannate compositions, preferably antihistamines, antitussives, decongestants, and expectorants. Such therapeutic agents may include tannate compounds and/or non-tannate compounds.

Antitussive agents are useful in the treatment of cough associated with upper respiratory conditions such as the common cold, respiratory infections, influenza, allergic rhinitis, perennial rhinitis, nasal and Eustachian tube congestion, and sinusitis.

The tannate salts of the opiate agents provide therapeutic activity for longer time periods. In effect, the inclusion of an active agent in a tannate salt form extends the release profile of the active agent and there is less spiking in pharmacological effect of the active agent. This leads to better compliance by the patient in that the active agent in the tannate salt form does not need to be given as often and there are fewer side effects, particularly from over-dosage effects.

The tannate compositions of the present invention can be made by methods known to those skilled in the art. Preparations of tannate compounds in a very pure form are taught in U.S. Pat. Nos. 5,599,846 and 5,663,415 to Chopdekar et al., which are herein incorporated in their entireties. In general, one method of making tannate compounds comprises reacting the base compound, such as chlorpheniramine or brompheniramine, with tannic acid in a solvent such as alcohol.

The compositions described herein are designed to be taken less frequently than non-tannate salt forms of the active opiate ingredient, such as twice a day in order to utilize the prolonged antitussive action of, for example, hydrocodone tannate. The opiate tannate compositions of the present invention may extend the effective release profile by as much as 50%, 150%, 200%, 250%, 300%, 350%, 400% or greater as compared to the non-tannate form of the opiate. The action of hydrocodone tannate may be utilized alone or in combination with the prolonged action of other compounds, either tannate or non-tannate in nature, or the immediate action of other compounds. The compositions of the present invention may be prepared for oral administration in the form of powders, capsules, elixirs, syrups and the preferred forms of tablets or suspensions. Administration by any other known route is also contemplated, such as transmucosally, transdermally, intravenously, intramuscularly or intraparenterally.

Tablets containing the unique hydrocodone tannate compositions of the present invention are prepared in a conventional manner by the addition of suitable pharmaceutical carriers including fillers, diluents, colorants, lubricants and the like, as well as conventional and well known binding and disintegrating agents. Chewable tablet formulations also include ingredients to enhance flavor and palatability such as sweeteners and natural and artificial flavors. Each tablet contains approximately 1 to 60 mg of hydrocodone tannate or approximately 1 to 120 mg of codeine tannate alone or in combination with a therapeutic amount of another pharmaceutical active ingredient. A typical chewable tablet composition of the present invention containing compressible sugar, magnesium stearate, microcrystalline cellulose (Avicel CE-15), citric acid, and flavor as described in Example 1 which follows, is prepared by well-known conventional tabletting techniques such as those disclosed in U.S. Pat. Nos. 3,018,221; 2,798,024 and 2,757,124.

### EXAMPLE 1

Hydrocodone Tannate/Brompheniramine Tannate Tablets

| Ingredient | Milligrams per Tablet |
|---|---|
| Hydrocodone tannate | 15.00 |
| Brompheniramine tannate | 6.00 |
| Compressible sugar, NF | 246.30 |
| Microcrystalline cellulose, NF | 30.00 |
| Citric acid, USP | 0.20 |
| Berry flavor | 0.90 |
| Magnesium Stearate, NF | 1.50 |

Tablets containing combinations of hydrocodone tannate and one or more additional active ingredients can comprise essentially the same ingredients in the same amounts. Changes in the additional active ingredient(s) present would be offset by the appropriate addition or subtraction to the compressible sugar amount. Total tablet weight would remain the same.

### EXAMPLE 2

Codeine Tannate Tablets

| Ingredient | Milligrams per Tablet |
|---|---|
| Codeine tannate | 30.00 |
| Compressible sugar, NF | 237.30 |
| Microcrystalline cellulose, NF | 30.00 |
| Citric acid, USP | 0.30 |
| Berry flavor | 0.90 |
| Magnesium Stearate, NF | 1.50 |

Tablets containing a combination of codeine tannate and one or more additional active ingredients would comprise essentially the same ingredients in the same amounts with the exception of the additional active ingredient(s) in place of the same amount by weight of compressible sugar.

Suspensions of the compositions of the present invention are prepared in a conventional manner such that each 5 mL (one teaspoon) would contain approximately 1 to 60 mg of hydrocodone tannate or 1 to 120 mg of codeine tannate alone or in combination with a therapeutic amount of another pharmaceutical active ingredient. Additionally, the suspension formulations may contain additional ingredients such as, but not limited to, citric acid, colorants, natural and artificial flavors, glycerin, magnesium aluminum silicate, methylparaben, propylparaben, purified water, sodium citrate, sweeteners such as sucralose, sucrose, or sorbitol, and xanthan gum. Example 2, which follows, is illustrative of a typical suspension formulation of the present invention prepared by conventional well-known compounding techniques.

### EXAMPLE 3

Hydrocodone Tannate Suspension

| Ingredient | Milligrams per 5 mL |
|---|---|
| Hydrocodone tannate | 15.00 |
| Brompheniramine tannate | 6.00 |
| Xanthan Gum, NF | 30.00 |
| Magnesium Aluminum Silicate, NF | 35.00 |
| Methylparaben, NF | 7.50 |
| Propylparaben, NF | 1.50 |
| Sucralose, NF | 7.50 |
| Glycerin, USP | 250.00 |
| Citric Acid, USP | 10.00* |
| Sodium Citrate, USP | 2.50* |
| Artificial Berry Flavor | 15.00 |
| FD&C Red #40 Dye | 0.20 |
| Purified Water, USP (Deionized) | adjust to 5 mL |

*Additional Citric Acid or Sodium Citrate may also be included in the formula if needed for pH adjustment.

Suspensions containing combinations of hydrocodone tannate and one or more additional active ingredients would comprise essentially the same ingredients in the same amounts. Changes in the additional active ingredient(s) present would be offset by the appropriate addition or subtraction to the purified water content.

### EXAMPLE 4

Codeine Tannate Suspension

| Ingredient | Milligrams per 5 mL |
|---|---|
| Codeine tannate | 30.00 |
| Xanthan Gum, NF | 30.00 |
| Magnesium Aluminum Silicate, NF | 35.00 |
| Methylparaben, NF | 7.50 |
| Propylparaben, NF | 1.50 |
| Sucralose, NF | 7.50 |
| Glycerin, USP | 250.00 |
| Citric Acid, USP | 10.00* |
| Sodium Citrate, USP | 2.50* |
| Artificial Berry Flavor | 15.00 |
| FD&C Red #40 Dye | 0.20 |
| Purified Water, USP (Deionized) | adjust to 5 mL |

*Additional Citric Acid or Sodium Citrate may also be included in the formula if needed for pH adjustment.

Suspensions containing a combination of hydrocodone tannate and one or more additional active ingredients would comprise essentially the same ingredients in the same amounts with the exception of the additional active ingredient(s) in place of the same amount by weight of purified water.

The dosage administered will be dependent on the mode of administration, the specific opiate tannate utilized, in addition to the age, health and weight of the recipient, kinds of concurrent treatment, if any, frequency of treatment and effect desired.

It should be understood that the above examples are illustrative of the exemplary modes only of the invention herein disclosed. Given the present disclosure, it is anticipated that numerous variations will occur to those skilled in the art. A latitude of modification, substitution and change is intended and in some instances, some features of the invention will be employed without a corresponding use of other features. Accordingly, it is intended that the spirit and scope of the invention disclosed herein should be limited only by the following claims.

What is claimed is:

1. A therapeutic composition for the symptomatic relief of cough associated with respiratory tract conditions such as the common cold, bronchial asthma, and acute and chronic bronchitis in warm-blooded animals in need of such treatment, said composition comprising a pharmaceutically effective amount of an opiate tannate and a pharmaceutically acceptable carrier.

2. The therapeutic composition of claim 1, wherein the opiate tannate is hydrocodone tannate.

3. The therapeutic composition of claim 1, wherein the pharmaceutically effective amount of an opiate tannate is about 1 to 60 mg of hydrocodone tannate.

4. The therapeutic composition of claim 1, wherein the pharmaceutically effective amount of an opiate tannate is about 15 mg of hydrocodone tannate.

5. The therapeutic composition of claim 1, wherein the opiate tannate is codeine tannate.

6. The therapeutic composition of claim 1, wherein the pharmaceutically effective amount of an opiate tannate is about 1 to 120 mg of codeine tannate.

7. The therapeutic composition of claim 1, wherein the pharmaceutically effective amount of an opiate tannate is about 30 mg of codeine tannate.

8. The therapeutic composition of claim 1, farther comprising an active ingredient selected from an antihistamine, a decongestant, an expectorant, an antitussant or combinations thereof.

9. The therapeutic composition of claim 1 in tablet form.

10. The therapeutic composition of claim 1 in suspension form.

11. A method for symptomatically treating and relieving the distress of cough associated with respiratory tract conditions resulting from the common cold, bronchial asthma, and acute and chronic bronchitis in warm-blooded animals, comprising orally administering to a warm-blooded animal in need of such treatment a therapeutically effective amount of a composition comprising a pharmaceutically effective amount of an opiate tannate and a pharmaceutically acceptable carrier.

12. The method of claim 11, wherein the opiate tannate is hydrocodone tannate.

13. The method of claim 11, wherein the pharmaceutically effective amount of an opiate tannate is about 1 to 60 mg of hydrocodone tannate.

14. The method of claim 11, wherein the pharmaceutically effective amount of an opiate tannate is about 15 mg of hydrocodone tannate.

15. The method of claim 11, wherein the opiate tannate is codeine tannate.

16. The method of claim 11, wherein the pharmaceutically effective amount of an opiate tannate is about 1 to 120 mg of codeine tannate.

17. The method of claim 11, wherein the pharmaceutically effective amount of an opiate tannate is about 30 mg of codeine tannate.

18. The method of claim 11, further comprising an active ingredient selected from an antihistamine, a decongestant, an expectorant, an antitussant or combinations thereof.

19. The method of claim 11, wherein the therapeutic composition is in tablet form.

20. The method of claim 11, wherein the therapeutic composition is in suspension form.
